(19) United States
(12) Patent Application Publication
(10) Pub. No.: US 2008/0146985 A1
(43) Pub. Date: Jun. 19, 2008

(54) Body treatment devices and methods

(76) Inventor: Jamie Glen House, Colorado Springs, CO (US)

Correspondence Address: MOAZZAM & ASSOCIATES, LLC, 7601 LEWINSVILLE ROAD, SUITE 304, MCLEAN, VA, 22102, US

(21) Appl. No.: 11/638,430

(22) Filed: Dec. 14, 2006

(51) Int. Cl. A61M 37/00

(52) U.S. Cl. 604/1

(57) ABSTRACT

Devices and methods are disclosed which aid to minimize or prevent infection by cleansing an exterior and interior portion of a body part simultaneously. The devices are similar to swabs but allow multi-function cleansing and/or sterilization in one step.

### BACKGROUND OF THE INVENTION

1. Field of the Invention

The present invention relates generally to body treatment devices and methods. More particularly, the present invention relates to devices and methods for cleaning, disinfecting and/or sterilizing body portions.

2. Background of the Invention

In home and hospital settings, it is common practice to cleanse the external surface of a portion of a body prior to insertion of a medical device, such as a catheter, tube, needle or other instrument. One of the major reasons for such external cleansing is to decrease or prevent the risk of infection of internal passages, tissues, organs or cavities. In body portions where the surface of the skin is initially unbroken prior to insertion, such external cleansing is typically effective to eliminate virtually any risk of infection. Such an example would be the cleansing of an insertion point for a needle prior to insertion for a shot or to withdraw blood. Such a surface would be, for example, an exterior surface of the arm. Since during the insertion process, the needle would make contact with the external surface of the arm that had just been cleansed, disinfected or sterilized, such needle would be effectively “clean” upon entering the tissue and the risk or likelihood of introducing contaminants into the body from the needle insertion would be minimal or nonexistent.

In body portions where there is no breakage of the skin or tissue from an exterior surface, and wherein an internal passage or cavity exists that is being used to introduce a medical device into the body, the prevention of infection becomes more complicated. One reason for this additional complication is that the body portion may have certain contaminants that may be tolerated at the locale where they are situated but would be problematic if and when introduced or transported to another locale within the body. One non-limiting example would be contaminants located at just inside the urethra. This portion of the body has been found to contain a large concentration of bacteria. A simple surface cleansing of the outer edge of the urethra may be effective in eliminating contaminants from the surface portion but is not effective in eliminating contaminants which may be positioned just inside or within the urethra. Likewise, such contaminants may be tolerated just inside the urethra because the skin cells are more able to withstand such contaminants at that point but when such contaminants are transported and introduced to other portions of the internal body, such as the bladder, such internal portions may not have the capacity to withstand the same contaminants in the same manner as the urethra. Thus, an infection ensues and medical complications arise.

Thus, there is a need in the healthcare industry for a cleansing technique or device that not only cleanses the external surface of a body portion prior to insertion of a medical device, but also a designated length of the passage into the body to also neutralize or destroy contaminants at such portions which are more prone to hosting such contaminants. Further, the technique or device should be easy to use and manufacture, effective and inexpensive so as to become widely used and accepted in treatment.

### SUMMARY OF THE INVENTION

The present invention provides techniques and devices which have the advantages of conventional devices but which overcome some of the drawbacks thereof. In certain exemplary embodiments, the present invention provides multi-planar cleansing, disinfecting and/or sterilizing of both the surface and a designated length of a body cavity or opening to prevent or eliminate contaminants which may be positioned on the immediate external surface or just inside of the interior cavity of the body portion being treated. The present invention thus provides a simple and effective method to cleanse and prepare a body portion prior to insertion of a medical device, which may be, for example, a catheter, tube or other instrument.

The present invention may be used in any “body portion,” which as defined herein and throughout this disclosure as any opening to an interior of the body. Non-limiting examples of body portions include, but are not limited to, urethral opening, meatus, anus, vagina, ear holes, and the like. One of ordinary skill in the art would be cognizant of the uses and applications of the present invention after consideration of the present disclosure. Such uses and application are within the purview of the present invention.

As used herein and throughout this disclosure, “contaminant” is defined as any substance that once introduced into the body or body tissue or body organ, has potential to cause infection or other harmful medical condition. Non-limiting examples of contaminants include, but are not limited to, bacteria, virus, debris, soil, dead skin, and the like. One having ordinary skill in the art would be cognizant of other such contaminants and such other contaminants are also within the purview of the present invention.

In one exemplary embodiment, the present invention is device for treatment of a body portion. The device includes an elongated shaft; a tip portion at one end of the shaft, wherein the tip portion contains an elongated tip at a distal end adjacent to a perpendicular separating wall; wherein the tip portion is insertable into a body portion.

In another exemplary embodiment, the present invention is device for treatment of an external and internal portion of a body part. The device includes an elongated shaft having an end; a tip portion at the end of the shaft, wherein the tip portion contains a guide wall which is perpendicular to the shaft and an elongation extending distal to the guide wall towards the end; wherein the tip portion is insertable into an internal portion of a body part.

In yet another exemplary embodiment, the present invention is a method for treatment of a body portion. The method includes providing an elongated shaft with a tip portion; making contact of the tip portion with an interior and exterior surface of the body portion simultaneously; wherein contact with the tip portion cleanses the body portion.

### BRIEF DESCRIPTION OF THE DRAWINGS

FIG. 1A shows a side view of a conventional technique of cleansing the outer surface of a penis with a treated cotton ball.

FIG. 1B shows a side view of the penis in FIG. 1A after cleansing of the external surface and prior to insertion of a medical device.

FIG. 1C shows a side view of the penis in FIG. 1C after the removal of the medical instrument, showing the spread of contaminants throughout the urethra and internal organs.

FIG. 2A shows a side view of a penis prior to application of an exemplary embodiment of the present invention.

FIG. 2B shows a side view of a device according to an exemplary embodiment of the present invention.

FIG. 2C shows a side view of the penis in FIG. 2A during use of the exemplary device shown in FIG. 2B, showing full removal or neutralization of contaminants throughout the critical region.

### DETAILED DESCRIPTION OF THE INVENTION

The present invention provides for devices and methods which prevent or eliminate the risk of contamination and infection of internal body passages and organs by cleaning, disinfecting and/or sterilizing a critical region most known to contain contaminants. In certain exemplary embodiments, an antiseptic swab using, for example, a betadine/iodeine or chlorhexadine, is presented which is structurally unique and performs a different function than conventional cotton balls or swabs. The current methods of cleaning before catheterization only involve cleansing the external meatus to sterilize the area which prevents pushing bacteria on the external meatus into the bladder. A major problem is that a very high bacterial load exist in the first ½ to 1 cm inside the internal urethra. Therefore, even though the external meatus/skin may be “sterile,” a sterile catheter pushes bacteria from the first part (½ to 1 cm) of the urethra into the bladder causing infection. This has been scientifically proven and is the focus of treatment of the present invention.

The present invention, in various embodiments, presents a unique swab that is partially shaped similar to conventional swabs but actually treats the outside area and an internal 1 cm (may be longer if needed) and serves to decrease infection during catheterization or other introduction of medical devices into the body. This present device may be used and be effective in other applications with internal bacterial load.

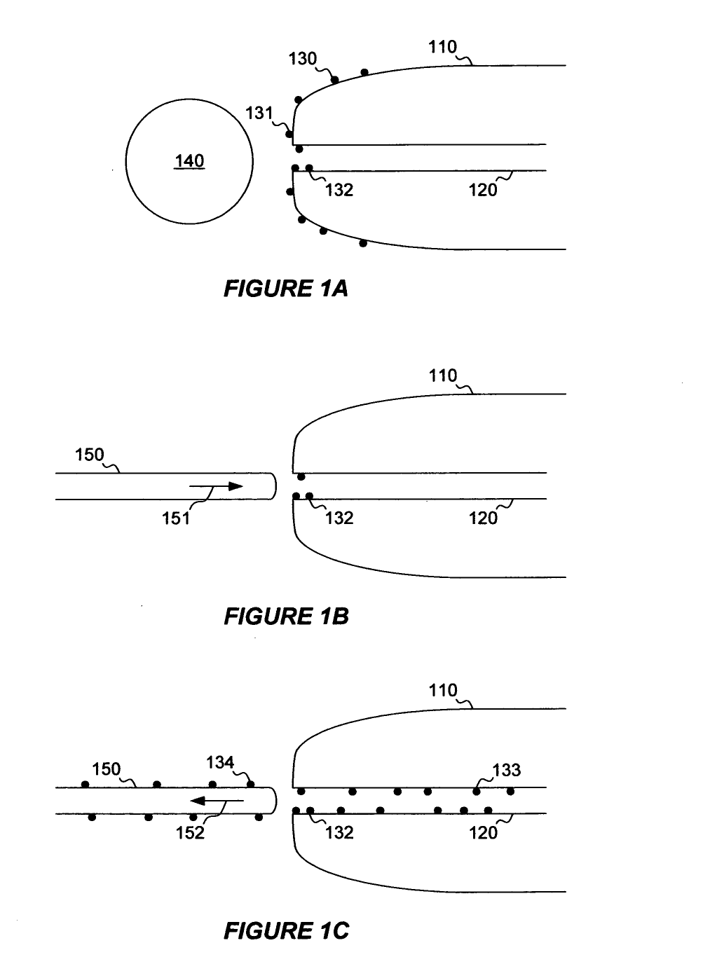

A conventional technique currently used to prepare a body part for insertion of a medical device is shown in FIG. 1A. In the example shown, cleansing of a tip of a penis is presented as an example of a body part being prepared for insertion of a medical device. However, it should be noted that other parts may be prepared conventionally in much the same manner as presented herein, and the present invention may be applied to such other parts as well, although only the use of the present invention with respect to a penis is shown for sake of simplicity. One having ordinary skill in the art would be cognizant of the use of present invention with such other parts.

In FIG. 1A, a penis 110 is shown prior to cleansing and preparing for insertion of a medical device, such as, for example, a catheter 150. Penis 110 has urethra 120 and is cleansed with a cotton ball 140 or swab (not shown) prior to insertion of a catheter 150. The external surface of penis 110 typically has bacterial or other contaminants 130 that may be easily cleaned or cleared with cotton ball 140 or swab or other cleansing or prepping pad. Contaminants 131 that are just exterior of the opening of the urethra 120 are most susceptible to creating infection in the urethral passage or bladder upon insertion of a catheter. These contaminants 131 may be cleaned or cleared with sufficient exposure to the cotton ball 140 or other swab or sheet. However, contaminants 132 that are just inside of the urethra 120 are out of reach of the cotton ball 140 or other tool conventionally used to cleanse and prepare the penis 110 before insertion of catheter 150.

As shown in FIG. 1B, when penis 110 is conventionally prepared and cleaned prior to insertion of catheter 150, contaminants 132 that are just inside the urethra 120 were unable to be cleared or cleaned during the preparation process. Thus, during insertion of catheter 150 in the direction 151 into urethra 120, contaminants 132 are further introduced or spread into the urethral passage and other internal organs resulting in spread of contaminants 133 and 134 in the internal organs and the surface of catheter, respectively, during extraction of the catheter in the direction of 152.

As shown in the sequence presented in FIGS. 1A-1C, although the exterior surface of penis 110 may be sufficiently cleared, cleaned or sterilized prior to insertion of a medical device 150, such conventional technique is not adequate enough to cleanse or clear contaminants 132 just within the urethral opening. Thus, this small remnant of contamination may be sufficient to cause infections and other medical problems when a catheter or other medical instrument is pushed through the urethra and carries with it this contaminant 132.

The present invention not only performs the surface cleansing function of conventional techniques but also addresses the contaminants that may be located just inside the urethra that cannot be cleansed or cleared using conventional techniques. This external surface and pre-designated length within the urethra (or other body cavity or tube) is an important source for bacteria or other contaminants and is the focus of the present invention.

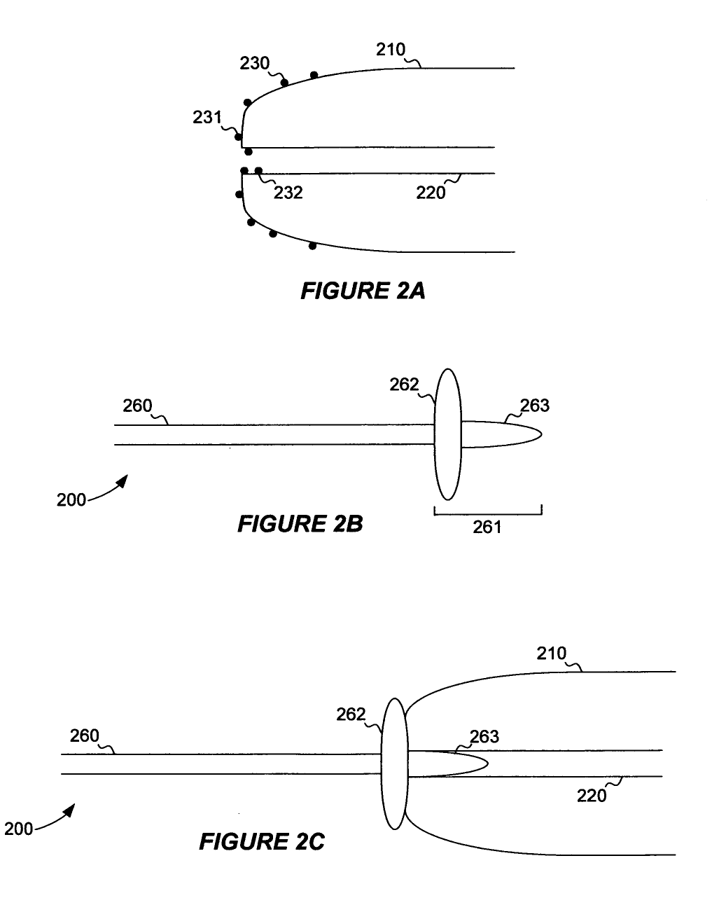

As shown in the exemplary embodiment presented in the series of FIGS. 2A-2C, penis 210 (or other body part having a small hole or foramen), has contaminants 230 on the exterior surface and just inside the opening of the urethra 220. The contaminants 231 that are closest to the opening or just inside 232 of the urethra 220 are most susceptible to be introduced into the urethra 220 and subsequent internal passages and organs, resulting in internal infection. Thus, it would be useful to cleanse, clear or sterilize contaminants that are near the urethral opening, both on the exterior and interior.

In one exemplary embodiment, as shown in FIG. 2B, device 200 may contain an elongated shaft 260 that facilitates holding, handling and directing tip portion 261 into a body opening, which may be a urethral opening, for example. The tip portion 261 has an elongated tip 263 that is separated from the elongated shaft 260 by a separator wall portion 262. The tip portion 261 may be composed of a material that is sterilized or contains sterilant, alcohol or other material that allows decontamination, disinfection, sterilization or other cleansing of contaminants. In one exemplary embodiment, the entire tip portion 261 may be covered with cotton or other material commonly used in conventional swabs, either pre-loaded with a liquid decontaminant or such that a user may apply a liquid to the tip portion 261 or the body portion prior to use.

In use, as shown in FIG. 2C, device 200 is shown with tip portion 261 positioned adjacent to tip end of penis 210. Separator wall 262 is in contact with tip of penis 210 and elongated tip 263 is within the interior of urethra 220. To cleanse contaminants 231 and 232 on the surface near the tip of penis 210 and just inside the urethra, respectively, handle 260 may be turned or pushed in and out of urethra to provide for adequate and effective contact between sterilant on the tip portion 261 and contaminants on or inside penis 210 in contact with tip portion 261. For added assurance of cleanliness, a cotton ball may be used to clean the outer surface of the penis, as shown in FIGS. 1A-1C, prior to application of the present invention, shown in FIG. 2C. The examples shown herein and throughout this disclosue use a male urethra for sake of simplicity but the invention is easily applicable to both male and female urethra.

The role of the present invention in cleansing, disinfecting and/or sterilizing the body portion depends in part on the substances used in treatment. For example, an antiseptic agent may be used including but not limited to: nitrofurazone, chlorhexadine, silver sulfadiazine, minocycline, rifampin, nystatin, fluconazole, miconazole, troconazole, ibutoconazole, or any combination thereof. Others are also possible.

Use of the present invention allows removal or neutralization of contaminants at locations of penis 210 that may contribute to or cause infection of the urethral passage, other internal passages or organs, for example, the bladder. Devices according to the present invention are designed to be disposable, simple to manufacture and use, effective and inexpensive. The length of the elongated tip 263 may be different according to what party portion is being treated, and the age and/or size of the body portion and or patient being treated. The separator wall 262 then acts as a stopping point and guide to the insertion length of the elongated tip 263 into the body portion.

In certain alternative embodiments, the separator wall 262 may be absent. That is, the operator would insert the narrow cotton swab into the initial 1-2 cm of the urethra. There would not be a safety perpendicular flange to stop insertion, but it could accomplish the cleansing procedure.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. A device for treatment of a body portion, the device comprising:
an elongated shaft;
a tip portion containing an absorbent material at one end of the shaft, wherein the tip portion contains an elongated tip at a distal end adjacent to a perpendicular separating wall; wherein the tip portion is insertable into a body portion until further insertion is prevented by the separating wall, which acts as a stopping point for insertion of the tip portion into the body portion.

2. The device of claim 1, wherein the tip portion is covered with material that can retain liquid.

3. The device of claim 2, wherein the liquid is de-contaminant.

4. The device of claim 3, wherein the liquid is a sterilant.

5. The device of claim 4, wherein the liquid is iodine.

6. The device of claim 1, wherein the tip portion can cleanse an outer surface and interior length of the body portion simultaneously during rotation of the shaft.

7. The device of claim 1, wherein the tip portion can cleanse an outer surface and interior portion of the body portion simultaneously during longitudinal motion along the length of the shaft.

8. A device for treatment of an external and internal portion of
a body part, the device comprising:
an elongated shaft having an end;
a tip portion having an absorbent material at the end of the shaft, wherein the tip portion contains a guide wall which is perpendicular to the shaft and an elongation extending distal to the guide wall towards the end; wherein the tip portion is insertable into an internal portion of a body part until the guide wall.

9. The device of claim 8, wherein the tip portion is covered with material that can retain liquid.

10. The device of claim 9, wherein the liquid is de-contaminant.

11. The device of claim 10, wherein the liquid is a sterilant.

12. The device of claim 11, wherein the liquid is iodine.

13. The device of claim 8, wherein the tip portion can cleanse an outer surface and interior length of the body portion simultaneously during rotation of the shaft.

14. The device of claim 8, wherein the tip portion can cleanse an outer surface and interior portion of the body portion simultaneously during longitudinal motion along the length of the shaft.

15. A method for treatment of a body portion, the method comprising:
providing an elongated shaft with a tip portion and an absorbent material;
making contact of the tip portion with an interior and exterior surface of the body portion simultaneously; wherein contact with the tip portion cleanses the body portion.

16. The method of claim 15, wherein the tip portion is covered with material that can retain liquid.

17. The method of claim 16, wherein the liquid is de-contaminant.

18. The method of claim 17, wherein the liquid is a sterilant.

19. The method of claim 15, wherein the contact is rotation of the shaft.

20. The device of claim 15, wherein the contact is longitudinal motion along the length of the shaft.
